(19) United States
(12) Patent Application Publication
(10) Pub. No.: US 2014/0017738 A1
(43) Pub. Date: Jan. 16, 2014

(54) SYSTEM FOR PRODUCING L-HOMOPHENYLALANINE AND A PROCESS FOR PRODUCING L-HOMOPHENYLALANINE

(71) Applicant: Universiti Sains Malaysia, Pulau Pinang (MY)

(72) Inventors: Abdul Latif AHMAD, Pulau Pinang (MY); Pei Ching OH, Pulau Pinang (MY); Syamsul Rizal ABD. SHUKOR, Pulau Pinang (MY)

(73) Assignee: Universiti Sains Malaysia, Palau Pinang (MY)

(21) Appl. No.: 14/010,957

(22) Filed: Aug. 27, 2013

Related U.S. Application Data
(62) Division of application No. 13/388,915, filed on Feb. 3, 2012.

(30) Foreign Application Priority Data
Jul. 6, 2009 (MY) PI20092840

(51) Int. Cl. C12P 13/04

(52) U.S. Cl. CPC C12P 13/04
USPC 435/106

(57) ABSTRACT

The present invention relates to a system for producing L-homophenylalanine and a process for producing L-homophenylalanine using the system. The system and the process include monitoring and controlling of the reaction conditions (e.g., temperature and pH) to desired or predetermined values. The monitoring, adjusting and agitating steps provided by the method thereby result in a more complete conversion of the available substrate and produce a sufficient yield of L-homophenylalanine.

### CROSS REFERENCE TO RELATED APPLICATIONS

This application is a Divisional of co-pending U.S. patent application Ser. No. 13/388,915, filed on Feb. 3, 2012, which is the U.S. national phase of International Application No. PCT/MY2010/000001, filed on Jan. 5, 2010, which claims priority to and benefit from Malaysian Patent Application No. PI20092840, filed on Jul. 6, 2009, all of which are incorporated herein by reference in their entirety.

### FIELD OF INVENTION

The present invention relates to a system for producing L-homophenylalanine and a process for producing L-homophenylalanine using the system.

### BACKGROUND

L-homophenylalanine ((S)-2-amino-4-pheny!butanoic acid) is extensively used in the pharmaceutical industry as a precursor for production of angiotensin-converting enzyme (ACE) inhibitors, which possess significant clinical application in the management of hypertension and congestive heart failure. Virtually all ACE inhibitors 15 with therapeutic significance such as enalapril, delapril, lisinopril, quinapril, ramipril, trandolapril, cilazapril and benzapril, refer to L-homophenylalanine as a common building block, due to the presence of L-homophenylalanine moiety as the central pharmacophore unit.

Chemical or biocatalytic route for L-homopheylalanine synthesis have been reported in various prior art documents. U.S. Pat. No. 6,146,859 discloses a process for producing L-homophenylalanine by reacting 2-oxo-4-pheylbutanoic acid with Lglutamic acid in the presence of tyrosine aminotransferase, and subsequently precipitating the L-homophenylalanine produced therefrom. However, the process requires genetically engineered tyrosine aminotransferase as the catalytic enzyme,

and high concentration of substrates.

Typical of prior techniques for producing L-homophenylalanine is the method disclosed by Bradshaw et al., Bioorganic Chemistry, 1 991, 19:29. Bradshaw reported a method of converting 2-oxo-4-phenylbutanoic acid to L-homophenylalanine by using Lphenylalanine dehydrogenase in the presence of cofactor. However, the authors 5 reported the use of conventional dialysis bag for the laboratory-scaled Lhomophenylalanine production. It was found that this process has low scale-up potential besides bearing several constraints in controlling the reaction conditions for optimum synthesis of product.

Senuma et al., Applied Biochemistry and Biotechnology, 1989, 22:141 reported a method of preparing L-homophenylalanine by converting 2-oxo-4-phenylbutanoic acid using microbial cells containing aminotransferase activity. Cho et al., Biotechnology and Bioengineering, 2003, 83:226 also synthesized the compound using a recombinant aromatic amino acid transaminase in the reaction media which permits efficient synthesis of L-homophenylalanine using a single transaminase reaction. Nevertheless, the aminotransferase activity is markedly inhibited by a high concentration of substrate in the reaction mixture leading to limitations in large-scale production.

Kao et al., Journal of Biotechnology, 2008, 134:231 are principally concerned with the production of L-homophenylalanine using recombinant Escherichia coli cells with dihydropyrimidinase and L-N-carbamoylase activities as whole cell biocatalysts. However, it was found that dihydropyrimidinase exhibited non-enantiospecificity for D,L-homophenylalanylhydantoin substrate, which needs to be improved in order to improve the yield of L-homophenylalanine.

Production of L-homophenylalanine as novel pharmaceutical intermediate has been studied for many years, as disclosed in the previous section, generating substantial literature and knowledge. Presently, laboratory bioreactors are used for the production of pharmaceutical drug precursors. Conventional laboratory bioreactors require separate and often complicated downstream processing for recovery or retention of isolated enzymes from the aqueous media.

It is clear from a review of the prior art processes for production of L-homophenylalanine that a hiatus exists with respect to techniques for in situ retention of biocatalysts when present in the reaction solution. While L-homophenylalanine has been produced either by selective retention of the biocatalysts in a dialysis bag or via a separate unit connected to the bioreactor system, in-situ configuration has not been implemented for L-homophenylalanine production, thus establishing the novelty of this invention.

### SUMMARY OF THE INVENTION

Accordingly, the present invention provides an integrated membrane bioreactor device with an acidification vessel system for producing L-homophenylalanine, the system includes (a) a vessel having an upper surface, lower surface and a plurality of side surfaces, said upper, lower, and side surfaces defining an interior body of said vessel, (b) a membrane holder and mesh for supporting a membrane at the lower surface of said vessel, (c) a port means for introduction of substrate, aqueous solution and cofactor into and removal from said interior body of said vessel, (d) a port means for introduction of at least a biocatalyst into said interior body of said vessel, (e) a means for introduction of an inert gas into said interior body of said vessel, (f) a reactor pressure transducer and a relief valve to control pressure in the vessel, (g) an outer jacket which surrounds said vessel for heating of fluid, (h) a means for monitoring and control of pH and temperature of solution in said vessel, (I) a port means for caustic dosing, (j) a stirrer, wherein the stirrer includes a driveshaft with a drive unit and impeller blades which are mounted on the shaft, (k) a vessel having an upper surface, lower surface and a plurality of side surfaces, said upper, lower, and side surfaces defining an interior body of said vessel, (I) a port means for introduction of a fluid from the vessel into said interior body of said vessel, (m) an outer jacket which surrounds said vessel for cooling of said fluid, (n) a means for monitoring and control of pH and temperature of solution in said vessel, (o) a port means for acid dosing and (p) a stirrer for agitation of reaction solution contained within said vessel. Furthermore, the present invention also provides a process for producing L-homophenylalanine using the integrated membrane bioreactor device with an acidification vessel system, the process includes the steps of (a) dissolving 2-oxo-4-phenylbutanoic acid, 1,4-dithiothreitol, sodium formate and NADH in deionized water at a pH of between 6 to 10 with an addition of a hydroxide, (b) adding L-phenylalanine dehydrogenase and formate dehydrogenase into a solution obtained from step (a), (C) stirring a solution obtained from step (b) at a temperature of between 27° C. to 5000 in an inert atmosphere, (d) separating and collecting of biocatalysts from a solution obtained from step (C), (e) acidifying a solution obtained from step (d), (f) filtering white precipitate obtained from step (e), (g) washing the white precipitate from step (f) with a non-reacting liquid and (h) drying the white -precipitate from step (g).

The present invention consists of several novel features and a combination of parts hereinafter fully described and illustrated in the accompanying description and the drawings, it being understood that various changes in the details may be made without departing from the scope of the invention or sacrificing any of the advantages of the present invention.

### BRIEF DESCRIPTION OF THE DRAWINGS

The present invention will be fully understood from the detailed description given herein below and the accompanying drawings which are given by way of illustration only, and thus are not limitative of the present invention, wherein:

FIG. 1 is a simplified schematic flow diagram of the integrated membrane bioreactor system for simultaneous reaction and retention of biocatalysts coupled to an acidification device for L-homophenylalanine production;

FIG. 2 is a schematic showing the synthesis of L-homophenylalanine (Compound 2) from 2-oxo-4phenylbutanoic acid (Compound 1) catalyzed by L-phenylalanine dehydrogenase coupled to NADH regeneration catalyzed by formate dehydrogenase; and

FIG. 3 is a HPLC graph, showing a measurement of the enantiomeric excess of the enzymatically synthesized L-homophenylalanine using Chiral T column.

### DESCRIPTION OF THE PREFERRED EMBODIMENTS

The present invention relates to a system for producing L-homophenylalanine and a process for producing L-homophenylalanine using the system. Hereinafter, this specification will describe the present invention according to the preferred embodiments of the present invention. However; it is to be understood that limiting the description to the preferred embodiments of the invention is merely to facilitate discussion of the present invention and it is envisioned that those skilled in the art may devise various modifications and equivalents without departing from the scope of the appended claims.

Generally, the present invention relates to a system (10) and a process for the production of L-homophenylalanine, in a reaction solution which occurs in a novel membrane bioreactor for simultaneous reaction and retention of biocatalysts. More particularly, the invention refers to an integrated membrane bioreactor for in situ reaction and selective retention of L-phenylalanine dehydrogenase and formate dehydrogenase for reuse, coupled to online control of pH and temperature to provide the optimal reaction condition for higher product yield. For this invention, the substrates, enzymes and coenzymes were pumped into the system (1 0) with inert atmosphere. The membrane bioreactor is incorporated with an ultrafiltration membrane with the appropriate molecular weight cutoff. Sufficient time was allowed for reaction to occur, and the product leaving the membrane bioreactor was subsequently acidified. The resulting white precipitate was collected, washed and dried in vacuum to yield Lhomophenylalanine without further purification. The present invention relates to a system (1 0) and a process for the production of Lhomophenylalanine by reacting 2-oxo-4-phenylbutanoic acid with 1,4-dithiothreito! and sodium formate in the presence of L-phenylalanine dehydrogenase, formate dehydrogenase and NADH cofactor in a reaction solution which occurs in a novel membrane bioreactor for simultaneous reaction and retention of biocatalysts. The invention relates to a system for in situ reaction and selective retention of Lphenylalanine dehydrogenase and formate dehydrogenase for reuse, also referred to as integrated membrane bioreactor.

The present invention makes use of a reactor module, coupled to an in situ separation unit for the continuous removal of products while retaining the biocatalysts. Membrane unit operations usually work under mild conditions and are environmental safe processes, as depicted in the synthesis of various pharmaceutical drug intermediates with low working temperature and pressure, with the additional advantage of minimum diffusional resistance due to direct contact between substrate and biocatalysts. For this invention, the substrates and the coenzyme were pumped into the membrane bioreactor, with the product leaving the membrane bioreactor unit through an ultrafiltration membrane with varying molecular weight cutoff for different enzymes. Enzymes are to be supplemented periodically dependent on the deactivation rates to the membrane bioreactor.

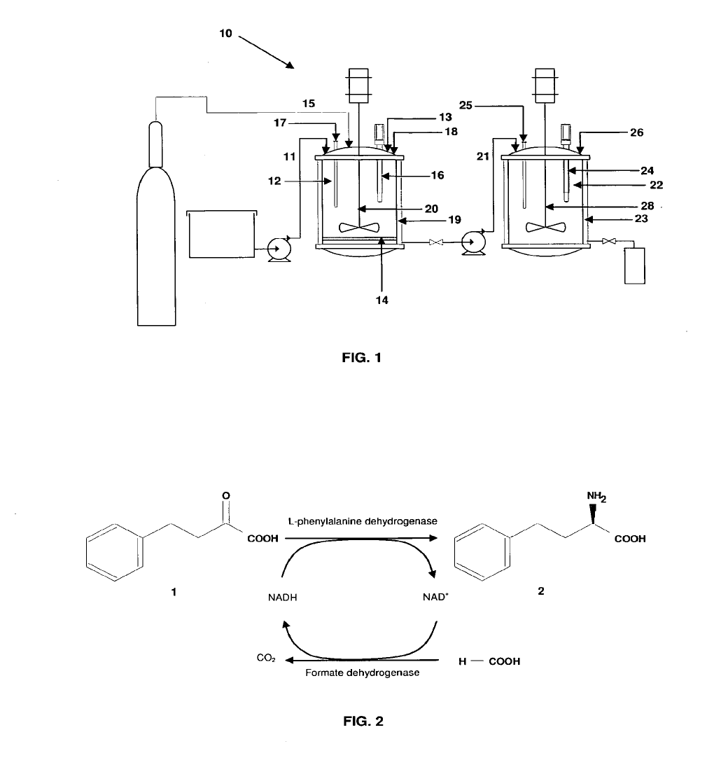

The system (10) of the invention comprises an integrated membrane bioreactor in which the reaction and separation of biocatalysts takes place, and in which the conditions and environment necessary for the reaction may be strictly controlled in an enclosed system, and a stirred reactor vessel for acidification of the product. The integrated membrane bioreactor serves as both reaction and separation vessel, thus invalidating the need for separate vessels for each function. A presently preferred embodiment of the current invention is provided in FIG. 1 in which the system (10) including an integrated membrane bioreactor device with an acidification device which produces L-homophenylalanine is described.

Hence, in a first embodiment of the present invention, there is provided the membrane bioreactor device for producing L-homophenylalanine. The device includes a first vessel (12) having an upper surface, lower surface and a plurality of side surfaces, said upper, lower, and side surfaces defining an interior body of said vessel (12), a membrane holder and mesh for supporting a membrane (14) at the lower surface of said vessel, a port means (11) for introduction of substrate, aqueous solution and cofactor into and removal from said interior body of said first vessel (12), a port means (13) for introduction of at least a biocatalyst into said interior body of said vessel, a means for introduction of an inert gas (15) into said interior body of said vessel, a15 reactor pressure transducer and a relief valve to control pressure in the vessel, an outer jacket (1 9) which surrounds said first vessel (12) for heating of fluid, means for monitoring and control of pH (16) and temperature (17) of solution in said first vessel (12), a port means (18) for caustic dosing and a stirrer (20), wherein the stirrer (20) includes a driveshaft with a drive unit and impeller blades which are mounted on the shaft.

In a second embodiment of the present invention, there is provided a device for acidifying and cooling of solution to produce L-homophenylalanine, the device includes a second vessel (22) having an upper surface, lower surface and a plurality of side surfaces, said upper, lower, and side surfaces defining an interior body of said second vessel (22), a port means (21) for introduction of a fluid from the first vessel (12) into said interior body of said second vessel (22), an outer jacket (23) which surrounds said second vessel (22) for cooling of said fluid, means for monitoring and control of pH (24) and temperature (25) of solution in said second vessel (22), a port means (26) for acid dosing and a stirrer (28) for agitation of reaction solution contained within said second vessel (22).

In a presently preferred embodiment of the current invention, both vessels (12, 22) are borosilicate glass cylindrical vessel. However, it may be appreciated that a tank of any suitable shape and any suitable material may be incorporated into the system of the present invention. The membrane holder is a loop of elastomer with a disc-shaped cross-section, designed to be seated in the groove at the lower surface of said vessel, preferably holding a stainless steel mesh to support the membrane (14). However, it may be appreciated that any design of membrane holder, a perforated or mesh screen of metal or any other suitable material currently used in the art, is envisioned in the design of the current invention.

Suitable reaction conditions for production of L-homophenylalanine, e.g., temperature, pH, concentration of biocatalysts and etc. are known in the art, but may vary in accordance with the particular drug precursor to be produced. Accordingly, it should be appreciated that the design of the present invention allows the condition of the reaction solution in both vessels to be monitored and suitably altered for controlling temperature, pH and the like, thus alleviating problem of unsteady state caused by manual regulation of reaction conditions. The system of the invention may also be equipped with one or more sampling ports for monitoring of the enzymatic process.

Advantages provided by the system described in the present invention include, but are not limited to:

1) Applicability to a wide range of substrates and biocatalysts;
2) Applicability to produce a wide range of drug precursors;
3) Ability to use said system for in situ reaction and retention of biocatalysts with reduction in moving parts and consequent ease of operation and reduction in capital and operating costs;
4) Use as multi-purpose vessel, including as a reactor or separation vessel;
5) Ability to monitor and control parameters (e.g. temperature by heating or cooling jacket and pH via automated caustic and acid dosing)

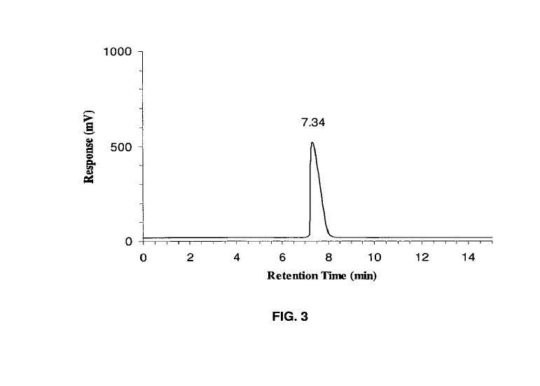

The process of producing L-homophenylalanine will now be described in detail with references to FIGS. 2 and 3. As shown in FIG. 2, the process is preferably conducted in a reaction mixture containing 2-oxo-4-phenylbutanoic acid, 1,4-dithiothreitol, sodium formate, formate dehydrogenase, NADH and L-phenylalanine dehydrogenase in deionized water. The foremost step comprises of dissolving 2-oxo-4-phenylbutanoic acid in deionized water containing 1,4-dithiothreitol, sodium formate and NADH at a pH of between 6 to 10. The reaction was initiated by adding L-phenylalanine dehydrogenase and formate dehydrogenase. The solution was stirred at appropriate temperature of between 27° C. to 50° C. and in an inert atmosphere with the addition of 1 ammonium hydroxide to maintain the pH at a constant value.

The reaction is carried out in a 1 L membrane bioreactor over a period of 1 week. The membrane bioreactor was equipped with an overhead stirrer, pH electrode connected to the data acquisition system, heating jacket, ports for caustic dosing and internal temperature monitoring using temperature sensor. The pH of the reaction solution was constantly monitored, and the system was connected directly to automated caustic dosing system to maintain the pH at optimum value. The same procedure as the above stated was applied in the case where varying solution temperature was achieved via heating using heating jacket. The -internal atmosphere was kept inert with argon gas. A flat sheet regenerated cellulose membrane with adequate molecular weight cutoff was incorporated for in situ separation and retention of biocatalysts. Upon completion of biotransformation and retention of the biocatalysts in the membrane bioreactor, the product enriched solution was acidified, preferably to pH 5.5 in the acidification vessel. The resulting white precipitate was collected by filtration, washed with cold water and dried in vacuum to yield L-homophenylalanine without further purification (>80% depending on the solution pH, temperature, amount of biocatalysts used, etc.).

The enantiomeric excess of L-homophenylalanine is ascertained using a chiral T column that shows an enantiomeric excess of over 99%. The chromatography is preferably carried out under the following conditions: Column, Astec Chirobiotic T; Flow rate, 1 ml/min; Eluents, ethanol/water=10/90 (v/v); and detector UV 210 nm. As shown in FIG. 3, the synthesized L-homophenylalanine had a retention time of 7.34 mm, where no 0-antipode could be observed. The product is optically pure as determined by optical rotation and compared to an authentic sample and literature values [a]=+480 (ci, 1 HCl).

What is claimed is:

1. A process for producing L-homophenylalanine comprising the steps of:
(a) dissolving 2-oxo-4-phenylbutanoic acid, 1,4-dithiothreitol, sodium formate and NADH in deionized water at a pH of between 6 to 10 with an addition of a hydroxide;
(b) adding L-phenylalanine dehydrogenase and formate dehydrogenase into a solution obtained from step (a);
(c) stirring a solution obtained from step (b) at a temperature of between 27° C. to 50° C. in an inert atmosphere;
(d) separating and collecting of biocatalysts from a solution obtained from step (c);
(e) acidifying a solution obtained from step (d);
(f) filtering white precipitate obtained from step (e);
(g) washing the white precipitate from step (f) with a non-reacting liquid; and
(h) drying the white precipitate from step (g).

2. The process as claimed in claim 1, wherein the hydroxide used in step (a) is ammonium hydroxide.

3. The process as claimed in claim 2, wherein ammonium hydroxide used is 1 N ammonium hydroxide.

4. The process as claimed in claim 1, wherein the solution in step (e) is acidified to pH 5.5 in the second vessel.

5. The process as claimed in claim 1, wherein the non-reacting liquid is water, preferably distilled water.

6. The process as claimed in claim 1, wherein step (h) is conducted in vacuum.

7. The process as claimed in claim 1, wherein said L-homophenylalanine has an enantiomeric excess of over 99%.
